(19) United States
(12) Patent Application Publication
(10) Pub. No.: US 2013/0104299 A1
(43) Pub. Date: May 2, 2013

(54) GOGGLES WITH A DISPLACEABLE TRANSPARENT FILM

(71) Applicant: Joy Chen, Tainan City (TW)

(72) Inventor: Joy Chen, Tainan City (TW)

(21) Appl. No.: 13/283,888

(22) Filed: Oct. 28, 2011

(51) Int. Cl. A61F 9/02

(52) U.S. Cl. 2/431

(57) ABSTRACT

Goggles include a body having a lens. A front face of the lens includes two side view sections corresponding to two eyes of a wearer and a central section corresponding to a nose of the wearer. At least one protrusion is formed on the central section and protrudes from the front face of the lens. A transparent film has a width larger than a width of the lens in a width direction. The transparent film is mounted to the front face of the lens and extends over the side view sections and the central section of the lens, with a distal end of the at least one protrusion abutting the transparent film. An actuating device is mounted to the body and coupled to the transparent film. The actuating device is operable to move the transparent film relative to the lens in the width direction.

### BACKGROUND OF THE INVENTION

The present invention relates to goggles with a displaceable transparent film and, more particularly, to goggles including a transparent film that can reliably be moved to provide enhanced use safety and enhanced use reliability.

Goggles are widely used to protect the human eyes in activities such as driving vehicles or sports involving high speed movements. Particularly, in addition to protection of the eyes, the goggles can eliminate adverse affect in racing sports to provide a clear view and safety. In sports involving outdoor off-road motorcycles and all-terrain vehicles, the road conditions are generally uneven and rugged. When the wheels move through mud, the mud may splash over the human body and the goggles worn by the driver. Thus, the driver can not see through the outer surface of the lens of the goggles stained with the mud. It is inconvenient and a waste of time and adversely affects the game result if the driver stops to wipe the mud off the lens.

In a solution to avoid the above situation, a displaceable transparent film 2′ is mounted to the front face 11′ of the lens 1′ of the goggles, as shown in FIGS. 7 and 7A. The transparent film 2′ has a width larger than that of the lens 1′ and can be moved by an actuating device 3′ having a coiling mechanism. Specifically, the coiling mechanism includes two reels 31′ and 32′ respectively on two sides of the front face 11′ of the lens 1′. Two ends of the transparent film 2′ are respectively attached to the reels 31′ and 32′. A portion of the transparent film 2′ can be moved from the reel 31′ to the other reel 32′ by operating a button 33′. When the transparent film 2′ is stained with mud or other dirt, the wearer can operate the button 33′ to move the stained section of the transparent film 2′ to the reel 32′ and to move a clean section of the transparent film 2′ to the front face 11′ of the lens 1′, providing a clear view. Thus, the wearer does not have to stop to clean the lens 1′. The vehicle speed and the game result are, thus, not adversely affected.

The transparent film 2′ completely abuts the front face 11′ of the lens 1′ under the action of the wind. The transparent film 2′ and the lens 1′ are generally made of plastic and, thus, have electrostatic adhering force therebetween. The engagement between the transparent film 2′ and the front face 11′ of the lens 1′ becomes firmer if the mud or dirt contains water. The transparent film 2′ can not displace easily or even get stuck when the button 33′ is operated, causing poor vision of the wearer moving at high speed and may even endangering the wearer. Ideally, a clear section of the transparent film 2′ should be moved to the front face 11′ of the lens 1′ and replace the stained section in 1 second. This is important in preventing long-term poor vision and unstable movement of the vehicle while the wearer is operating the button 33′ with his or her hand.

FIG. 8 shows an improved transparent film 2′ by providing a plurality of through-holes 21′ in the transparent film 2′ to reduce the friction and electrostatic adherence between the transparent film 2′ and the front face 11′ of the lens 1′ by reducing the contact area therebetween. However, the mud or dirt may splash over the front face 11′ of the lens 1′ via the through-holes 21′. Furthermore, the contact area between the contact area between the transparent film 2′ and the front face 11′ of the lens 1′ is still relatively large. Namely, the above disadvantage can not be effectively mitigated. Furthermore, formation of the through-holes by drilling increases the manufacturing costs.

FIG. 9 shows another option for smoothly moving the transparent film 2′ by providing a lubricant 5′ on the transparent film 2′. However, the transmission property of the transparent film 2′ is reduced by the lubricant 5′. Application of the lubricant 5′ also increases the manufacturing costs. Furthermore, the lubricant 5′ is liable to leak or deteriorate due to contact with water or after a period of time.

### BRIEF SUMMARY OF THE INVENTION

Goggles according to the present invention include a body having a lens with a front face. The front face of the lens includes two side view sections corresponding to two eyes of a wearer and a central section corresponding to a nose of the wearer. The lens has a width in a width direction. At least one protrusion is formed on the central section and protrudes from the front face of the lens. The at least one protrusion has a distal end. A transparent film has a width larger than the width of the lens. The transparent film is mounted to the front face of the lens and extends over the two side view sections and the central section of the lens, with the distal end of the at least one protrusion of the lens abutting the transparent film. An actuating device is mounted to the body and coupled to the transparent film. The actuating device is operable to move the transparent film relative to the lens in the width direction.

The at least one protrusion can be integrally formed with the lens as a single, monolithic piece. Alternatively, the at least one protrusion can be originally separate from the lens and bonded to the central section of the front face of the lens.

The distal end of the at least one protrusion can be arcuate.

In an example, a plurality of spaced protrusions is provided on the central section of the front face of the lens and each extends along a height of the central section of the lens in a height direction perpendicular to the width direction.

The lens can be curved or a parallelepiped.

The present invention will become clearer in light of the following detailed description of illustrative embodiments of this invention described in connection with the drawings.

### DESCRIPTION OF THE DRAWINGS

FIG. 1 shows a perspective view of goggles with a displaceable transparent film according to the present invention.

FIG. 2 shows a cross sectional view taken along section line A-A of FIG. 1.

FIG. 2A shows an enlarged view of a circled portion of FIG. 2.

FIG. 3 shows a cross sectional view taken along section line B-B of FIG. 1.

FIG. 3A shows an enlarged view of a circled portion of FIG. 3.

FIG. 4 shows a cross sectional view similar FIG. 2, illustrating movement of the transparent film.

FIG. 5 shows an enlarged view of a portion of the goggles of FIG. 4.

FIG. 6 shows a cross sectional view of goggles of another example according to the present invention, with the lens being plane.

FIG. 6A shows an enlarged view of a circled portion of FIG. 6.

FIG. 7 shows a cross sectional view of a goggle with a conventional film.

FIG. 7A illustrates an enlarged view of a circled portion of FIG. 7.

FIG. 8 shows a partial, cross sectional view of a lens and another conventional film.

FIG. 9 shows a partial, cross sectional view of a lens and a further conventional film.

### DETAILED DESCRIPTION OF THE INVENTION

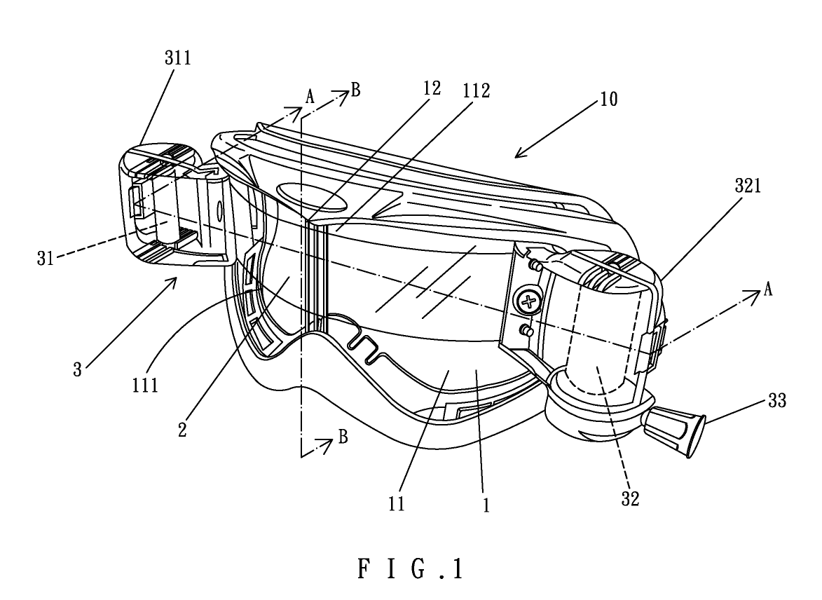

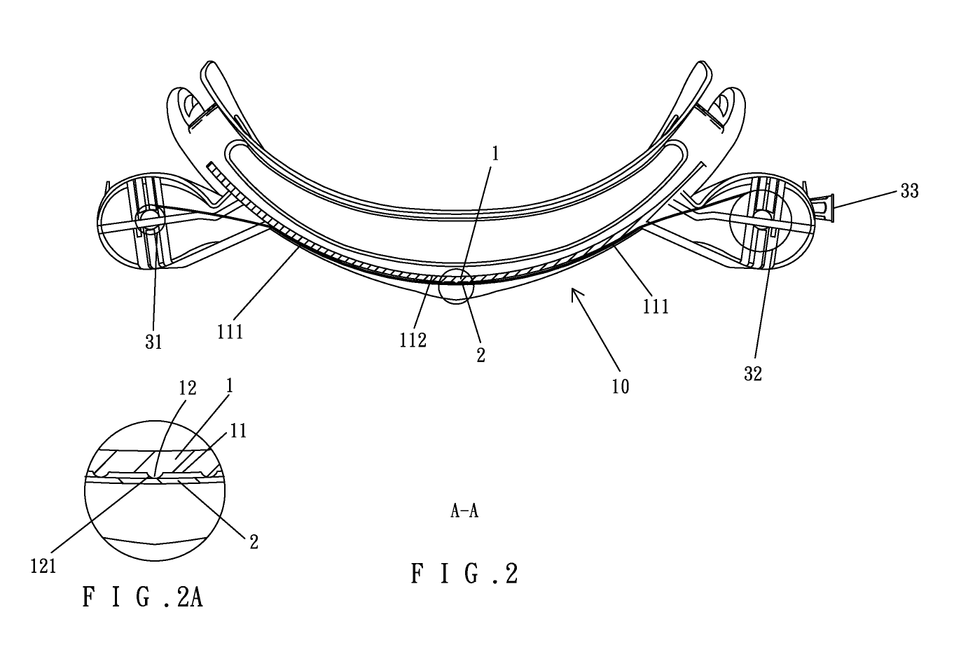

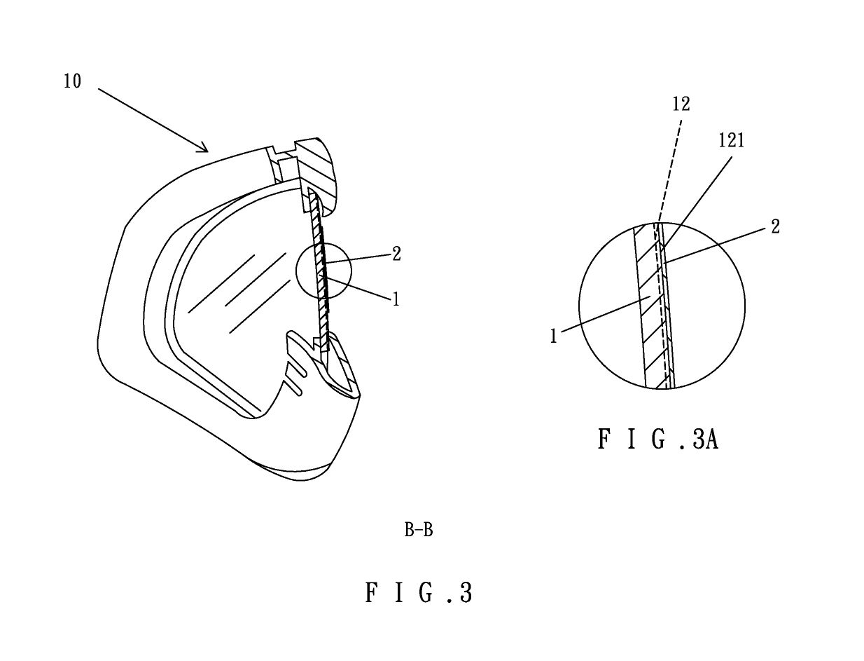

With reference to FIGS. 1, 2, 2A, 3, and 3A, goggles according to the present invention include a body 10, a transparent film 2, and an actuating device 3. The body 10 includes a lens 1 having a front face 11. The front face 11 of the lens 1 includes two side view sections 111 corresponding to two eyes of a wearer and a central section 112 corresponding to a nose of the wearer. The lens 1 has a width in a width direction. At least one protrusion 12 is formed on the central section 112 and protrudes from the front face 11 of the lens 1. In the example shown, the lens 1 is curved, and three protrusions 12 are integrally formed with the lens 1 as a single, monolithic piece and spaced from each other. Each protrusion 12 is in the form of a ridge extends along a height of the central section 112 of the lens 1 in a height direction perpendicular to the width direction and includes an arcuate distal end 121 (FIG. 2A).

The transparent film 2 has a width larger than the width of the lens 1. The transparent film 2 is mounted to the front face 11 of the lens 1 and extends over the side view sections 111 and the central section 112 of the lens 1. The distal ends 121 of the protrusions 12 of the lens 1 abut the transparent film 2.

The actuating device 3 is mounted to the body 1 and located on two sides of the lens 1. The actuating device 3 is coupled to the transparent film 2 and operable to move the transparent film 2 relative to the lens 1 in the width direction. The displacement of the transparent film 2 is substantially the same as the width of the lens 1. In the example shown, the actuating device 3 includes two reels 31 and 32 respectively on two sides of the front face 11 of the lens 1. Two ends of the transparent film 2 are respectively attached to the reels 31 and 32. A button 33 is provided on the reel 32 and can be operated to move the transparent film 2.

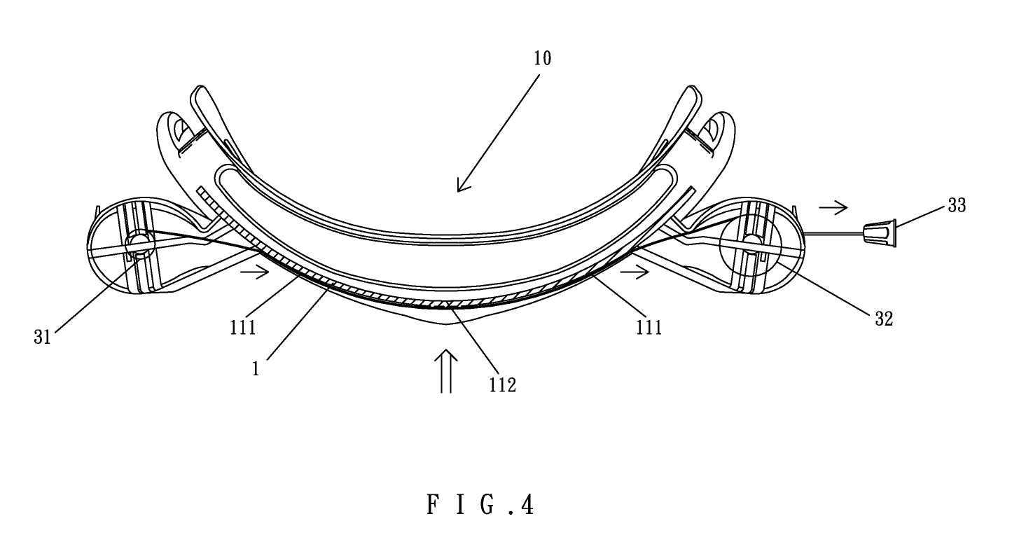

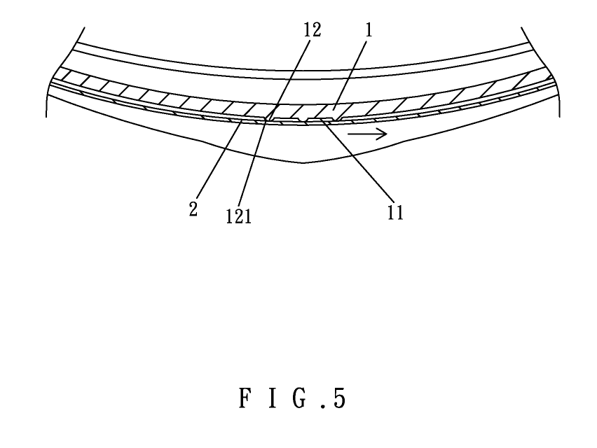

With reference to FIGS. 4 and 5, when the transparent film 2 is moved by operating the button 33, the transparent film 2 contacts the front face 11 of the lens 1 by the distal ends 121 of the protrusions 12. Thus, the friction between the transparent film 2 and the lens 1 is significantly reduced. Furthermore, the electrostatic adherence between the transparent film 2 and the front face 11 of the lens 1 can be reduced. Further, the displacement of the transparent film 2 relative to the lens 1 is easy even if the transparent film 2 is stained with water. Thus, the actuating device 3 can smoothly and rapidly move the transparent film 2, providing enhanced safety. Furthermore, the central section 112 of the lens 1 protrudes forward from the body 10 such that the protrusions 12 on the central section 112 are the main contact section subjected to force while the transparent film 2 is moved by the actuating device 3. Namely, the side view sections 111 of the front face 11 of the lens 1 would not be subjected to force from the transparent film 2, allowing rapid displacement of the transparent film 2.

The protrusions 12 of the lens 1 of the goggles according to the present invention are free of the disadvantage of loss of the lubricant in the conventional transparent film. Furthermore, the transmission property of the transparent film 2 is not adversely affected by the protrusions 12 of the lens 1. The transparent film 2 can be stably and reliably moved even after long-term use. Furthermore, the lens 1 can be integrally formed to form the protrusions 12 without the need of additional working hours for formation or of application of the lubricant. The manufacturing cost is almost not increased. Furthermore, the protrusions 12 are located on the central section 112 of the lens 1 without adversely affecting the view provided by the side view sections 111, maintaining excellent transmission of the goggles.

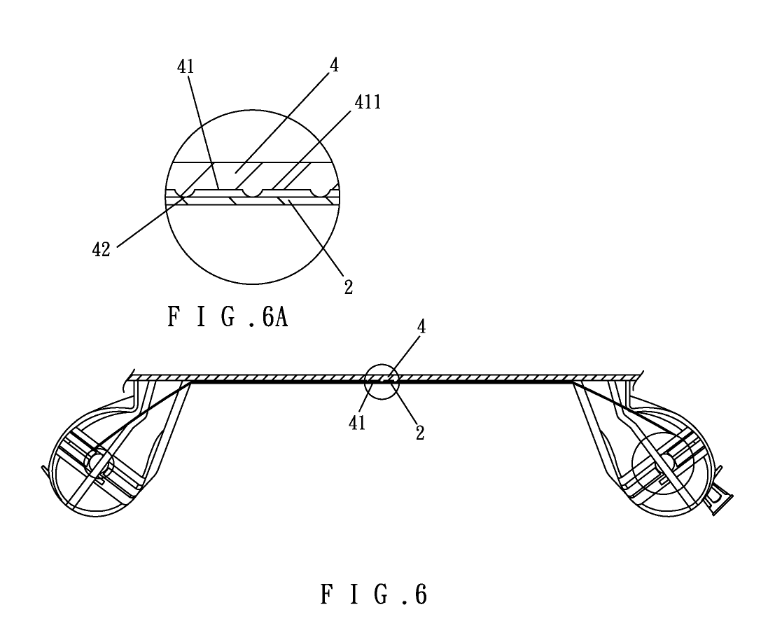

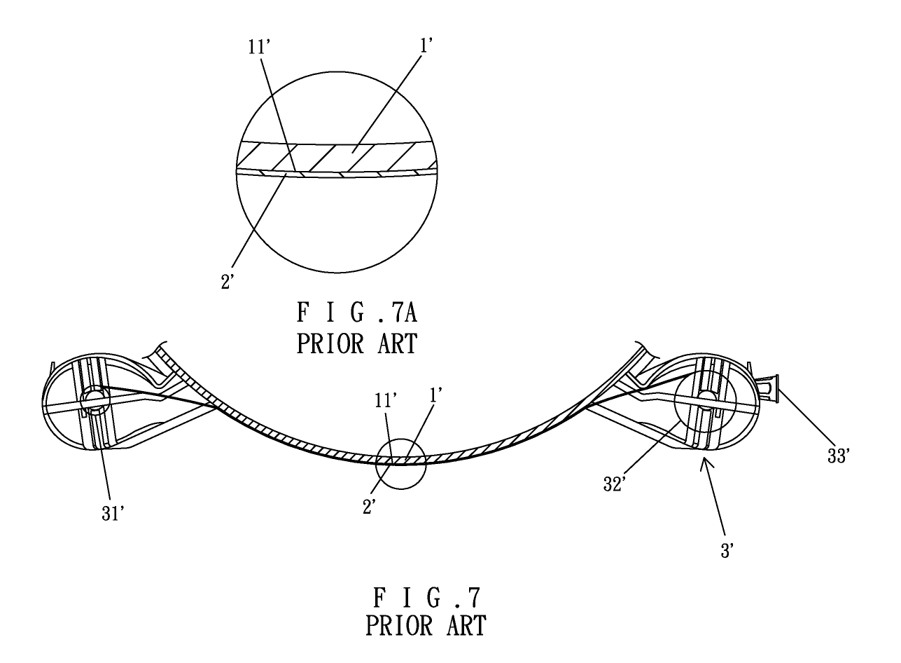

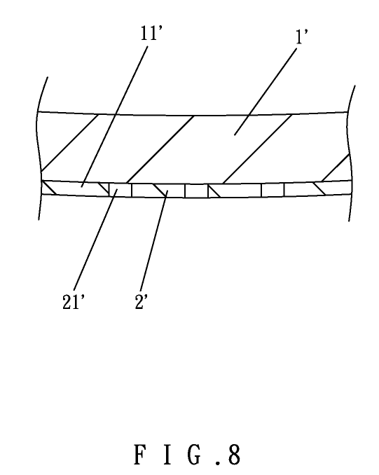

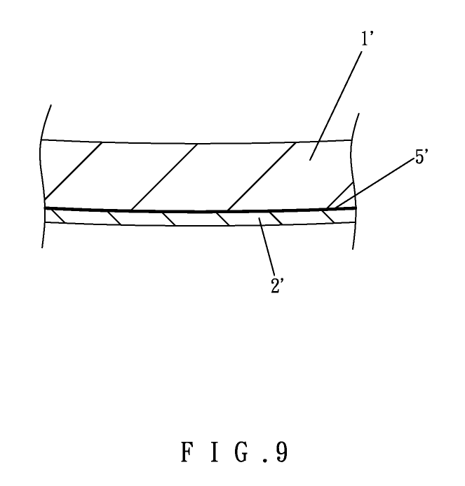

With reference to FIG. 6 showing another example of the goggles according to the present invention, the lens 1 can be a parallelepiped and includes protrusions 42 on the central section 411 of the front surface 41 of the lens 4. The transparent film 2 is in contact with the protrusions 42 to reduce the friction during displacement of the transparent film 2. Thus, the transparent film 2 can be moved smoothly and rapidly.

The total area of the distal ends 121 of the protrusions 12 can be smaller than one twentieth of the area of the front face 11 of the lens 1 to effectively reduce the friction during movement of the transparent film 2. A clean section of the transparent film 2 can be moved in a short period of time (such as within 1 second) by controlling the actuating device 3 when the transparent film 2 is stained with mud or other dirt while the vehicle is moving. The time required by the wearer to operate the actuating device 3 can be reduced, eliminating unstable movement of the vehicle and effectively assuring use safety.

The protrusions 12 can be in the form of knurls. Furthermore, the protrusions 12 can be originally separate from the lens 1 and bonded to the central section 112 of the front face 11 of the lens 1. Furthermore, the protrusions 12 can be spaced from each other in the height direction of the lens 1. Additional protrusions can be provided on the two side view sections 111 if the additional protrusions do not adversely affect the vision of the wearer.

The goggles according to the present invention provide enhanced safety and enhanced reliability almost without increasing the cost.

Although specific embodiments have been illustrated and described, numerous modifications and variations are still possible without departing from the essence of the invention. The scope of the invention is limited by the accompanying claims.

1. Goggles comprising:
a body including a lens having a front face, with the front face of the lens including two side view sections corresponding to two eyes of a wearer and a central section corresponding to a nose of the wearer, with the lens having a width in a width direction, with at least one protrusion formed on the central section and protruding from the front face of the lens, with the at least one protrusion having a distal end;
a transparent film having a width larger than the width of the lens, with the transparent film mounted to the front face of the lens and extending over the two side view sections and the central section of the lens, with the distal end of the at least one protrusion of the lens abutting the transparent film; and
an actuating device mounted to the body and coupled to the transparent film, with the actuating device operable to move the transparent film relative to the lens in the width direction.

2. The goggles as claimed in claim 1, with the at least one protrusion integrally formed with the lens as a single, monolithic piece.

3. The goggles as claimed in claim 1, with the at least one protrusion separate from the lens, with the at least one protrusion bonded to the central section of the front face of the lens.

4. The goggles as claimed in claim 1, with the distal end of the at least one protrusion being arcuate.

5. The goggles as claimed in claim 4, the at least one protrusions including a plurality of protrusions spaced from each other, with each of the plurality of protrusions extending along a height of the central section of the lens in a height direction perpendicular to the width direction.

6. The goggles as claimed in claim 4, with the lens being curved or a parallelepiped.
